(19) United States
(12) Patent Application Publication
(10) Pub. No.: US 2006/0184065 A1
(43) Pub. Date: Aug. 17, 2006

(54) Method and apparatus for storing an analyte sampling and measurement device

(76) Inventors: Ajay Deshmukh, San Francisco, CA (US); Ganapati Mauze, Sunnyvale, CA (US); Dirk Boecker, Palo Alto, CA (US)

Correspondence Address: HELLER EHRMAN LLP, 275 MIDDLEFIELD ROAD, MENLO PARK, CA, 94025-3506, US

(21) Appl. No.: 11/352,385

(22) Filed: Feb. 10, 2006

Related U.S. Application Data
(60) Provisional application No. 60/652,316, filed on Feb. 10, 2005.

(51) Int. Cl. A61B 5/00; A61M 1/00

(52) U.S. Cl. 600/583; 604/318

(57) ABSTRACT

Methods and apparatus are provided for storing used and unused test strips in a desiccated environment. In one embodiment, the method comprises providing an analyte sampling device having a instrument housing and a cartridge having a plurality of penetrating members wherein the penetrating members are slidably movable to extend outward from lateral openings on the cartridge to penetrate tissue, where the sampling device include a plurality of analyte sensing members. The device is designed to use a cassette that will fit inside the device but also contain the cartridge in a desiccated environment. The user may open a lid or access door on the cassette to allow for lancing and sample capture. The lid is closed to re-establish a sealed condition inside the cassette once lancing is complete.

### CROSS-REFERENCE TO RELATED APPLICATIONS

This application claims the benefit of U.S. Ser. No. 60/652,316, filed Feb. 10, 2005, which application is fully incorporated herein by reference.

### BACKGROUND OF THE INVENTION

1. Technical Field

The technical field relates to analyte sampling devices, and more specifically, methods and devices for storing analyte sampling and measurement devices in a safe, usable condition.

2. Background Art

Lancing devices are known in the medical health-care products industry for piercing the skin to produce blood for analysis. Typically, a drop of blood for this type of analysis is obtained by making a small incision in the fingertip, creating a small wound, which generates a small blood droplet on the surface of the skin.

Early methods of lancing included piercing or slicing the skin with a needle or razor. Current methods utilize lancing devices that contain a multitude of spring, cam and mass actuators to drive the lancet; These include cantilever springs, diaphragms, coil springs, as well as gravity plumbs used to drive the lancet. The device may be held against the skin and mechanically triggered to ballistically launch the lancet. Unfortunately, the pain associated with each lancing event using known technology discourages patients from testing. In addition to vibratory stimulation of the skin as the driver impacts the end of a launcher stop, known spring based devices have the possibility of firing lancets that harmonically oscillate against the patient tissue, causing multiple strikes due to recoil. This recoil and multiple strikes of the lancet is one major impediment to patient compliance with a structured glucose monitoring regime.

Success rate generally encompasses the probability of producing a blood sample with one lancing action, which is sufficient in volume to perform the desired analytical test. The blood may appear spontaneously at the surface of the skin, or may be “milked” from the wound. Milking generally involves pressing the side of the digit, or in proximity of the wound to express the blood to the surface. In traditional methods, the blood droplet produced by the lancing action must reach the surface of the skin to be viable for testing.

When using existing methods, blood often flows from the cut blood vessels but is then trapped below the surface of the skin, forming a hematoma. In other instances, a wound is created, but no blood flows from the wound. In either case, the lancing process cannot be combined with the sample acquisition and testing step. Spontaneous blood droplet generation with current mechanical launching system varies between launcher types but on average it is about 50% of lancet strikes, which would be spontaneous. Otherwise milking is required to yield blood. Mechanical launchers are unlikely to provide the means for integrated sample acquisition and testing if one out of every two strikes does not yield a spontaneous blood sample.

Many diabetic patients (insulin dependent) are required to self-test for blood glucose levels five to six times daily. The large number of steps required in traditional methods of glucose testing ranging from lancing, to milking of blood, applying blood to the test strip, and getting the measurements from the test strip discourages many diabetic patients from testing their blood glucose levels as often as recommended. Tight control of plasma glucose through frequent testing is therefore mandatory for disease management. The pain associated with each lancing event further discourages patients from testing. Additionally, the wound channel left on the patient by known systems may also be of a size that discourages those who are active with their hands or who are worried about healing of those wound channels from testing their glucose levels.

Another problem frequently encountered by patients who must use lancing equipment to obtain and analyze blood samples is the amount of manual dexterity and hand-eye coordination required to properly operate the lancing and sample testing equipment due to retinopathies and neuropathies particularly, severe in elderly diabetic patients. For those patients, operating existing lancet and sample testing equipment can be a challenge. Once a blood droplet is created, that droplet must then be guided into a receiving channel of a small test strip or the like. If the sample placement on the strip is unsuccessful, repetition of the entire procedure including re-lancing the skin to obtain a new blood droplet is necessary.

Early methods of using test strips required a relatively substantial volume of blood to obtain an accurate glucose measurement. This large blood requirement made the monitoring experience a painful one for the user since the user may need to lance deeper than comfortable to obtain sufficient blood generation. Alternatively, if insufficient blood is spontaneously generated, the user may need to “milk” the wound to squeeze enough blood to the skin surface. Neither method is desirable as they take additional user effort and may be painful. The discomfort and inconvenience associated with such lancing events may deter a user from testing their blood glucose levels in a rigorous manner sufficient to control their diabetes.

A further impediment to patient compliance is the technique for storing these analyte sampling and analyte detecting devices. The devices used to measure analyte levels are typically stored in a humidity controlled or other safe environment to maintain the device shelf life. This often involves using a variety of containers, some for the test strips and some for the lancets. The introduction of multiple storage devices and the cumbersome design may discourage users from keeping their equipment in a usable condition, further degrading user test compliance and measurement accuracy.

### SUMMARY OF THE INVENTION

Accordingly, an object of the present invention is to provide an improved fluid sampling device.

Another object of the present invention is to provide a fluid sampling device, and its methods of use, that provides a desiccated case for the entire instrument housing.

Yet another object of the present invention is to provide a fluid sampling device, and its methods of use, that includes a plurality of analyte detection members, a plurality of penetrating members, and a desiccant that is external to the plurality of penetrating members.

A further object of the present invention is to provide a fluid sampling device, and its methods of use, that includes a plurality of analyte detection members, a plurality of penetrating members, a desiccant that is external to the plurality of penetrating members and holds the desiccant.

These and other objects of the present invention are achieved in, a fluid sampling device that has an instrument housing. A cartridge defines a plurality of cavities. The cartridge is sized to fit within the instrument housing. A cassette houses the cartridge and is sized to fit within the instrument housing. A plurality of penetrating members are at least partially contained in the cavities of the cartridge. The penetrating members are slidably movable to extend outward from the cartridge to penetrate tissue. The cavities each have a longitudinal opening that provides access to an elongate portion of the penetrating member. A sterility barrier is coupled to the cartridge. The sterility barrier covers a plurality of the longitudinal openings. The sterility barrier covers the lateral openings and is configured to be moved so that the elongate portion may be accessed by the gripper without touching the barrier. Desiccant material is inside the device to reduce humidity therein.

In another embodiment of the present invention, a device is provided for use in penetrating tissue to obtain a body fluid sample. An instrument housing and a cartridge are provided. A plurality of penetrating members are slidably coupled to the cartridge. Each penetrating member has a distal end sufficiently sharp to pierce tissue and is moveable relative to the other ones of the penetrating members, so that the distal end of the respective penetrating member is movable to penetrate tissue. Each penetrating member is a bare lancet that does not penetrate an outer sterility barrier during actuation. A plurality of analyte sensing members are mounted about the instrument housing. A cassette contains the cartridge and is sized to fit within the instrument housing. The cassette provides a sealed environment when a lid on the cassette is closed to improve the storage condition of the analyte sensing members. A desiccant is in the device.

In another embodiment of the present invention, a method provides an analyte sampling device having a instrument housing and a cartridge with a plurality of penetrating members. The penetrating members are slidably movable to extend outward from lateral openings on the cartridge to penetrate tissue. The cartridge is in a sealed cassette that contains desiccant. Te cassette has a lid that is opened when the cartridge is about to be used.

### BRIEF DESCRIPTION OF THE DRAWINGS

FIG. 1 illustrates one embodiment of a fluid sampling device of the present invention.

FIGS. 2(a) and 2(b) illustrate embodiments of displacement and velocity profiles, respectively, of a harmonic spring/mass powered driver.

FIG. 2(c) illustrates an embodiment of a controlled displacement profile of a penetrating member driver.

FIG. 2(d) illustrates an embodiment of a the controlled velocity profile of a penetrating member driver.

FIG. 3 illustrates an embodiment of a fluid sampling device of the present invention with a feedback loop.

FIG. 4 illustrates an embodiment of a tissue penetration device of the present invention that has a lancing device with a controllable driver coupled to a tissue penetration element.

FIG. 5 illustrates in greater detail a lancing device of the present invention.

FIG. 6 illustrates one embodiment of a fluid sampling device of the present invention that has a cartridge which can be removably inserted into an apparatus for driving penetrating members to pierce skin or tissue.

FIG. 7 illustrates an embodiment of a fluid sampling device of the present invention.

FIG. 8 illustrates an embodiment of a fluid sampling device of the present invention with a disc assembled with test strips and desiccant.

FIG. 9 illustrates the FIG. 8 embodiment with a cassette.

FIG. 10 illustrates an embodiment of a fluid sampling device of the present invention with a cassette that has a sealed environment.

FIG. 11 illustrates an embodiment of a fluid sampling device of the present invention with a door or lid swung to an open position where the user can access a test strip and provide body fluid sample for analysis.

FIG. 12 illustrates an embodiment of a fluid sampling device of the present invention with a cassette housed inside of the device.

FIG. 13 illustrates an embodiment of a fluid sampling device of the present invention where a front end is incorporated on the outside of a more square cassette.

### DESCRIPTION OF THE SPECIFIC EMBODIMENTS

The present invention provides a solution for body fluid sampling. Specifically, some embodiments of the present invention provide improved devices and methods for storing a sampling device. The invention may use a high density penetrating member design. It may use penetrating members of smaller size, such as but not limited to diameter or length, than those of conventional penetrating members known in the art. The device may be used for multiple lancing events without having to remove a disposable from the device. The invention may provide improved sensing capabilities. At least some of these and other objectives described herein will be met by embodiments of the present invention.

It is to be understood that both the foregoing general description and the following detailed description are exemplary and explanatory only and are not restrictive of the invention, as claimed. It may be noted that, as used in the specification and the appended claims, the singular forms “a”, “an” and “the” include plural referents unless the context clearly dictates otherwise. Thus, for example, reference to “a material” may include mixtures of materials, reference to “a chamber” may include multiple chambers, and the like. References cited herein are hereby incorporated by reference in their entirety, except to the extent that they conflict with teachings explicitly set forth in this specification.

In this specification and in the claims which follow, reference will be made to a number of terms which shall be defined to have the following meanings:

“Optional” or “optionally” means that the subsequently described circumstance may or may not occur, so that the description includes instances where the circumstance occurs and instances where it does not. For example, if a device optionally contains a feature for analyzing a blood sample, this means that the analysis feature may or may not be present, and, thus, the description includes structures wherein a device possesses the analysis feature and structures wherein the analysis feature is not present.

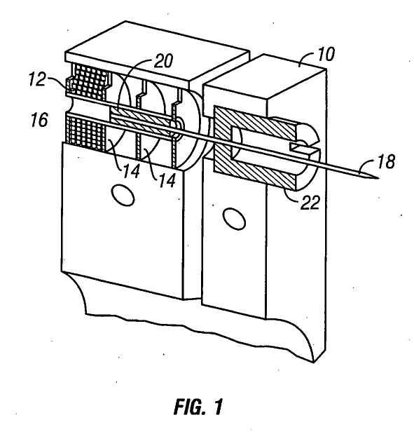

The present invention may be used with a variety of different penetrating member drivers. It is contemplated that these penetrating member drivers may be spring based, solenoid based, magnetic driver based, nanomuscle based, or based on any other mechanism useful in moving a penetrating member along a path into tissue. It should be noted that the present invention is not limited by the type of driver used with the penetrating member feed mechanism. One suitable penetrating member driver for use with the present invention is shown in FIG. 1. This is an embodiment of a solenoid type electromagnetic driver that is capable of driving an iron core or slug mounted to the penetrating member assembly using a direct current (DC) power supply. The electromagnetic driver includes a driver coil pack that is divided into three separate coils along the path of the penetrating member, two end coils and a middle coil. Direct current is alternated to the coils to advance and retract the penetrating member. Although the driver coil pack is shown with three coils, any suitable number of coils may be used, for example, 4, 5, 6, 7 or more coils may be used.

Referring to the embodiment of FIG. 1, the stationary iron housing 10 may contain the driver coil pack with a first coil 12 flanked by iron spacers 14 which concentrate the magnetic flux at the inner diameter creating magnetic poles. The inner insulating housing 16 isolates the penetrating member 18 and iron core 20 from the coils and provides a smooth, low friction guide surface. The penetrating member guide 22 further centers the penetrating member 18 and iron core 20. The penetrating member 18 is protracted and retracted by alternating the current between the first coil 12, the middle coil, and the third coil to attract the iron core 20. Reversing the coil sequence and attracting the core and penetrating member back into the housing retracts the penetrating member. The penetrating member guide 22 also serves as a stop for the iron core 20 mounted to the penetrating member 18.

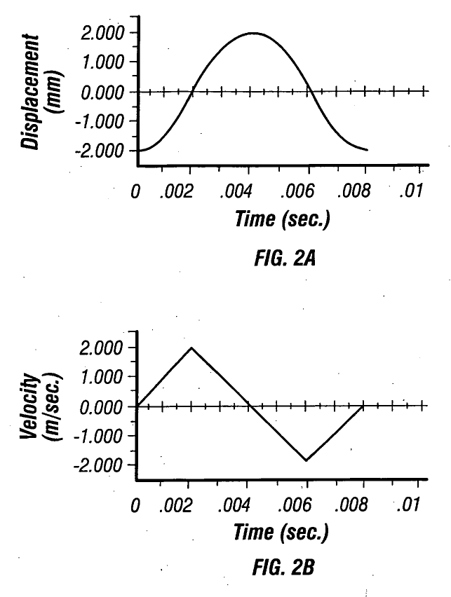

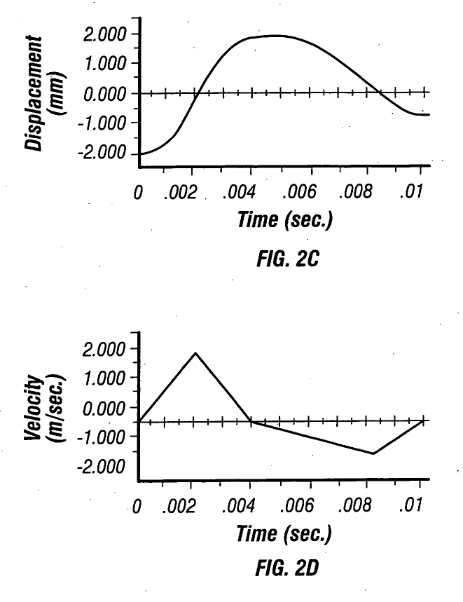

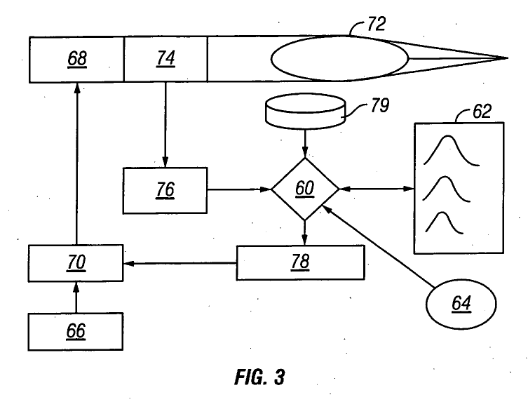

As discussed above, tissue penetration devices which employ spring or cam driving methods have a symmetrical or nearly symmetrical actuation displacement and velocity profiles on the advancement and retraction of the penetrating member as shown in FIGS. 2(a) through 2(d) and 3. In most of the available lancet devices, once the launch is initiated, the stored energy determines the velocity profile until the energy is dissipated. Controlling impact, retraction velocity, and dwell time of the penetrating member within the tissue can be useful in order to achieve a high success rate while accommodating variations in skin properties and minimize pain. Advantages can be achieved by taking into account of the fact that tissue dwell time is related to the amount of skin deformation as the penetrating member tries to puncture the surface of the skin and variance in skin deformation from patient to patient based on skin hydration.

In this embodiment, the ability to control velocity and depth of penetration may be achieved by use of a controllable force driver where feedback is an integral part of driver control. Such drivers can control either metal or polymeric penetrating members or any other type of tissue penetration element. The dynamic control of such a driver is illustrated in FIG. 2(c) which illustrates an embodiment of a controlled displacement profile and FIG. 2(d) which illustrates an embodiment of a the controlled velocity profile. These are compared to FIGS. 2(a) and 2(b), which illustrate embodiments of displacement and velocity profiles, respectively, of a harmonic spring/mass powered driver. Reduced pain can be achieved by using impact velocities of greater than about 2 m/s entry of a tissue penetrating element, such as a lancet, into tissue. Other suitable embodiments of the penetrating member driver are described in commonly assigned, copending U.S. patent application Ser. No. 10/127,395, (Attorney Docket No. 38187-2551) filed Apr. 19, 2002 and previously incorporated herein.

FIG. 3 illustrates the operation of a feedback loop using a processor 60. The processor 60 stores profiles 62 in non-volatile memory. A user inputs information 64 about the desired circumstances or parameters for a lancing event. The processor 60 selects a driver profile 62 from a set of alternative driver profiles that have been preprogrammed in the processor 60 based on typical or desired tissue penetration device performance determined through testing at the factory or as programmed in by the operator. The processor 60 may customize by either scaling or modifying the profile based on additional user input information 64. Once the processor has chosen and customized the profile, the processor 60 is ready to modulate the power from the power supply 66 to the penetrating member driver 68 through an amplifier 70. The processor 60 may measure the location of the penetrating member 72 using a position sensing mechanism 74 through an analog to digital converter 76 linear encoder or other such transducer. Examples of position sensing mechanisms have been described in the embodiments above and may be found in the specification for commonly assigned, copending U.S. patent application Ser. No. 10/127,395, (Attorney Docket No. 38187-2551) filed Apr. 19, 2002 and previously incorporated herein. The processor 60 calculates the movement of the penetrating member by comparing the actual profile of the penetrating member to the predetermined profile. The processor 60 modulates the power to the penetrating member driver 68 through a signal generator 78, which may control the amplifier 70 so that the actual velocity profile of the penetrating member does not exceed the predetermined profile by more than a preset error limit. The error limit is the accuracy in the control of the penetrating member.

After the lancing event, the processor 60 can allow the user to rank the results of the lancing event. The processor 60 stores these results and constructs a database 80 for the individual user. Using the database 79, the processor 60 calculates the profile traits such as degree of painlessness, success rate, and blood volume for various profiles 62 depending on user input information 64 to optimize the profile to the individual user for subsequent lancing cycles. These profile traits depend on the characteristic phases of penetrating member advancement and retraction. The processor 60 uses these calculations to optimize profiles 62 for each user. In addition to user input information 64, an internal clock allows storage in the database 79 of information such as the time of day to generate a time stamp for the lancing event and the time between lancing events to anticipate the user's diurnal needs. The database stores information and statistics for each user and each profile that particular user uses.

In addition to varying the profiles, the processor 60 can be used to calculate the appropriate penetrating member diameter and geometry suitable to realize the blood volume required by the user. For example, if the user requires about 1-5 microliter volume of blood, the processor 60 may select a 200 micron diameter penetrating member to achieve these results. For each class of lancet, both diameter and lancet tip geometry, is stored in the processor 60 to correspond with upper and lower limits of attainable blood volume based on the predetermined displacement and velocity profiles.

The lancing device is capable of prompting the user for information at the beginning and the end of the lancing event to more adequately suit the user. The goal is to either change to a different profile or modify an existing profile. Once the profile is set, the force driving the penetrating member is varied during advancement and retraction to follow the profile. The method of lancing using the lancing device comprises selecting a profile, lancing according to the selected profile, determining lancing profile traits for each characteristic phase of the lancing cycle, and optimizing profile traits for subsequent lancing events.

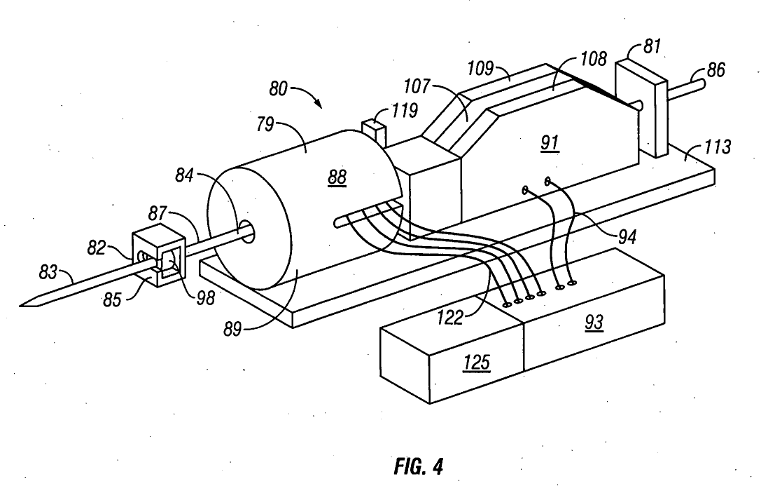

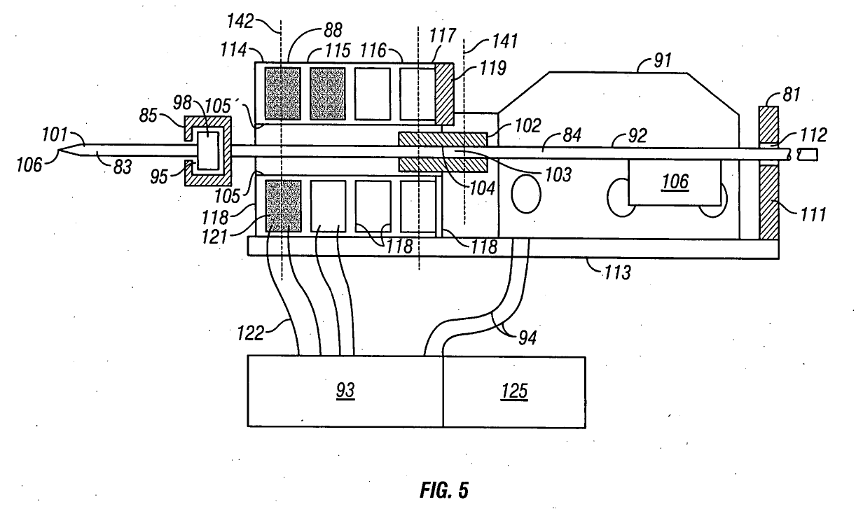

FIG. 4 illustrates an embodiment of a tissue penetration device, more specifically, a lancing device 80 that includes a controllable driver 179 coupled to a tissue penetration element. The lancing device 80 has a proximal end 81 and a distal end 82. At the distal end 82 is the tissue penetration element in the form of a penetrating member 83, which is coupled to an elongate coupler shaft 84 by a drive coupler 85. The elongate coupler shaft 84 has a proximal end 86 and a distal end 87. A driver coil pack 88 is disposed about the elongate coupler shaft 84 proximal of the penetrating member 83. A position sensor 91 is disposed about a proximal portion 92 of the elongate coupler shaft 84 and an electrical conductor 94 electrically couples a processor 93 to the position sensor 91. The elongate coupler shaft 84 driven by the driver coil pack 88 controlled by the position sensor 91 and processor 93 form the controllable driver, specifically, a controllable electromagnetic driver.

Referring to FIG. 5, the lancing device 80 can be seen in more detail, in partial longitudinal section. The penetrating member 83 has a proximal end 95 and a distal end 96 with a sharpened point at the distal end 96 of the penetrating member 83 and a drive head 98 disposed at the proximal end 95 of the penetrating member 83. A penetrating member shaft 201 is disposed between the drive head 98 and the sharpened point 97. The penetrating member shaft 201 may be comprised of stainless steel, or any other suitable material or alloy and have a transverse dimension of about 0.1 to about 0.4 mm. The penetrating member shaft may have a length of about 3 mm to about 50 mm, specifically, about 15 mm to about 20 mm. The drive head 98 of the penetrating member 83 is an enlarged portion having a transverse dimension greater than a transverse dimension of the penetrating member shaft 201 distal of the drive head 98. This configuration allows the drive head 98 to be mechanically captured by the drive coupler 85. The drive head 98 may have a transverse dimension of about 0.5 to about 2 mm.

A magnetic member 102 is secured to the elongate coupler shaft 84 proximal of the drive coupler 85 on a distal portion 203 of the elongate coupler shaft 84. The magnetic member 102 is a substantially cylindrical piece of magnetic material having an axial lumen 204 extending the length of the magnetic member 102. The magnetic member 102 has an outer transverse dimension that allows the magnetic member 102 to slide easily within an axial lumen 105 of a low friction, possibly lubricious, polymer guide tube 105′ disposed within the driver coil pack 88. The magnetic member 102 may have an outer transverse dimension of about 1.0 to about 5.0 mm, specifically, about 2.3 to about 2.5 mm. The magnetic member 102 may have a length of about 3.0 to about 5.0 mm, specifically, about 4.7 to about 4.9 mm. The magnetic member 102 can be made from a variety of magnetic materials including ferrous metals such as ferrous steel, iron, ferrite, or the like. The magnetic member 102 may be secured to the distal portion 203 of the elongate coupler shaft 84 by a variety of methods including adhesive or epoxy bonding, welding, crimping or any other suitable method.

Proximal of the magnetic member 102, an optical encoder flag 206 is secured to the elongate coupler shaft 84. The optical encoder flag 206 is configured to move within a slot 107 in the position sensor 91. The slot 107 of the position sensor 91 is formed between a first body portion 108 and a second body portion 109 of the position sensor 91. The slot 107 may have separation width of about 1.5 to about 2.0 mm. The optical encoder flag 206 can have a length of about 14 to about 18 mm, a width of about 3 to about 5 mm and a thickness of about 0.04 to about 0.06 mm.

The optical encoder flag 206 interacts with various optical beams generated by LEDs disposed on or in the position sensor body portions 108 and 109 in a predetermined manner. The interaction of the optical beams generated by the LEDs of the position sensor 91 generates a signal that indicates the longitudinal position of the optical flag 206 relative to the position sensor 91 with a substantially high degree of resolution. The resolution of the position sensor 91 may be about 200 to about 400 cycles per inch, specifically, about 350 to about 370 cycles per inch. The position sensor 91 may have a speed response time (position/time resolution) of 0 to about 120,000 Hz, where one dark and light stripe of the flag constitutes one Hertz, or cycle per second. The position of the optical encoder flag 206 relative to the magnetic member 102, driver coil pack 88 and position sensor 91 is such that the optical encoder 91 can provide precise positional information about the penetrating member 83 over the entire length of the penetrating member's power stroke.

An optical encoder that is suitable for the position sensor 91 is a linear optical incremental encoder, model HEDS 9200, manufactured by Agilent Technologies. The model HEDS 9200 may have a length of about 20 to about 30 mm, a width of about 8 to about 12 mm, and a height of about 9 to about 11 mm. Although the position sensor 91 illustrated is a linear optical incremental encoder, other suitable position sensor embodiments could be used, provided they posses the requisite positional resolution and time response. The HEDS 9200 is a two channel device where the channels are 90 degrees out of phase with each other. This results in a resolution of four times the basic cycle of the flag. These quadrature outputs make it possible for the processor to determine the direction of penetrating member travel. Other suitable position sensors include capacitive encoders, analog reflective sensors, such as the reflective position sensor discussed above, and the like.

A coupler shaft guide 111 is disposed towards the proximal end 81 of the lancing device 80. The guide 111 has a guide lumen 112 disposed in the guide 111 to slidingly accept the proximal portion 92 of the elongate coupler shaft 84. The guide 111 keeps the elongate coupler shaft 84 centered horizontally and vertically in the slot 102 of the optical encoder 91.

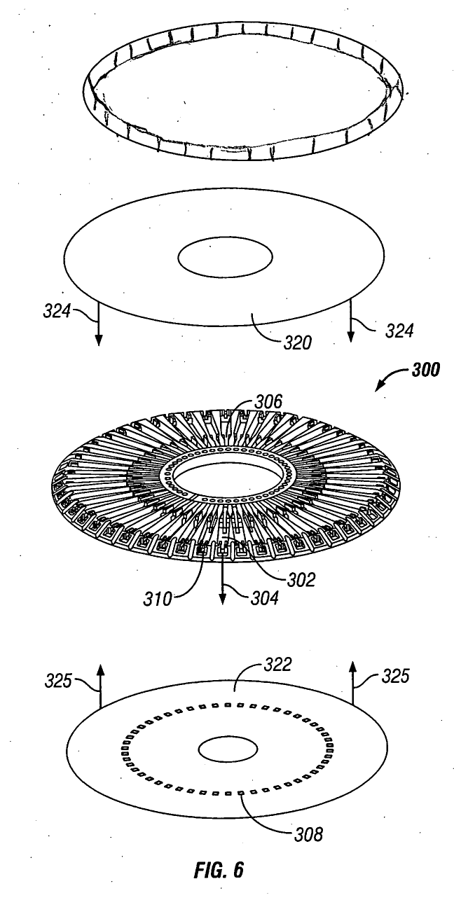

Referring now to FIG. 6, a still further embodiment of a cartridge according to the present invention will be described. FIG. 6 shows one embodiment of a cartridge 300 which may be removably inserted into an apparatus for driving penetrating members to pierce skin or tissue: The cartridge 300 has a plurality of penetrating members 302 that may be individually or otherwise selectively actuated so that the penetrating members 302 may extend outward from the cartridge, as indicated by arrow 304, to penetrate tissue. In the present embodiment, the cartridge 300 may be based on a flat disc with a number of penetrating members such as, but in no way limited to, (25, 50, 75,100, . . . ) arranged radially on the disc or cartridge 800. It should be understood that although the cartridge 300 is shown as a disc or a disc-shaped housing, other shapes or configurations of the cartridge may also work without departing from the spirit of the present invention of placing a plurality of penetrating members to be engaged, singly or in some combination, by a penetrating member driver.

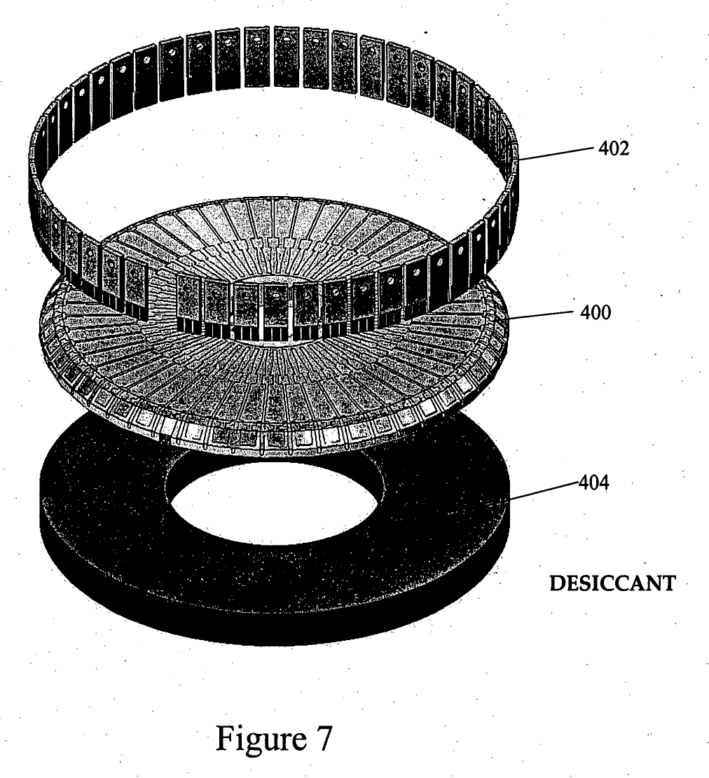

Each penetrating member 302 may be contained in a cavity 306 in the cartridge 300 with the penetrating member's sharpened end facing radially outward and may be in the same plane as that of the cartridge. The cavity 306 may be molded, pressed, forged, or otherwise formed in the cartridge. Although not limited in this manner, the ends of the cavities 306 may be divided into individual fingers (such as one for each cavity) on the outer periphery of the disc. The particular shape of each cavity 306 may be designed to suit the size or shape of the penetrating member therein or the amount of space desired for placement of the analyte sensing members 808. For example and not limitation, the cavity 306 may have a V-shaped cross-section, a U-shaped cross-section, C-shaped cross-section, a multi-level cross section or the other cross-sections. The opening 810 through which a penetrating member 302 may exit to penetrate tissue may also have a variety of shapes, such as but not limited to, a circular opening, a square or rectangular opening, a U-shaped opening, a narrow opening that only allows the penetrating member to pass, an opening with more clearance on the sides, a slit, a configuration as shown in FIG. 7, or the other shapes.

In this embodiment, after actuation, the penetrating member 302 is returned into the cartridge and may be held within the cartridge 300 in a manner so that it is not able to be used again. By way of example and not limitation, a used penetrating member may be returned into the cartridge and held by the launcher in position until the next lancing event. At the time of the next lancing, the launcher may disengage the used penetrating member with the cartridge 300 turned or indexed to the next clean penetrating member such that the cavity holding the used penetrating member is position so that it is not accessible to the user (i.e. turn away from a penetrating member exit opening). In some embodiments, the tip of a used penetrating member may be driven into a protective stop that hold the penetrating member in place after use. The cartridge 300 is replaceable with a new cartridge 300 once all the penetrating members have been used or at such other time or condition as deemed desirable by the user.

Referring still to the embodiment in FIG. 6, the cartridge 300 may provide sterile environments for penetrating members via seals, foils, covers, polymeric, or similar materials used to seal the cavities and provide enclosed areas for the penetrating members to rest in. In the present embodiment, a foil or seal layer 320 is applied to one surface of the cartridge 300. The seal layer 320 may be made of a variety of materials such as a metallic foil or other seal materials and may be of a tensile strength and other quality that may provide a sealed, sterile environment until the seal layer 320 is penetrate by a suitable or penetrating device providing a preselected or selected amount of force to open the sealed, sterile environment. Each cavity 306 may be individually sealed with a layer 320 in a manner such that the opening of one cavity does not interfere with the sterility in an adjacent or other cavity in the cartridge 800. As seen in the embodiment of FIG. 6, the seal layer 320 may be a planar material that is adhered to a top surface of the cartridge 800.

Depending on the orientation of the cartridge 300 in the penetrating member driver apparatus, the seal layer 320 may be on the top surface, side surface, bottom surface, or other positioned surface. For ease of illustration and discussion of the embodiment of FIG. 6, the layer.320 is placed on a top surface of the cartridge 800. The cavities 306 holding the penetrating members 302 are sealed on by the foil layer 320 and thus create the sterile environments for the penetrating members. The foil layer 320 may seal a plurality of cavities 306 or only a select number of cavities as desired.

In a still further feature of FIG. 6, the cartridge 300 may optionally include a plurality of analyte sensing members 308 on a substrate 822 which may be attached to a bottom surface of the cartridge 300. The substrate may be made of a material such as, but not limited to, a polymer, a foil, or other material suitable for attaching to a cartridge and holding the analyte sensing members 308. As seen in FIG. 6, the substrate 322 may hold a plurality of analyte sensing members, such as but not limited to, about 10-50, 50-100, or other combinations of analyte sensing members. This facilitates the assembly and integration of analyte sensing members 308 with cartridge 300. These analyte sensing members 308 may enable an integrated body fluid sampling system where the penetrating members 302 create a wound tract in a target tissue, which expresses body fluid that flows into the cartridge for analyte detection by at least one of the analyte sensing members 308. The substrate 322 may contain any number of analyte sensing members 308 suitable for detecting analytes in cartridge having a plurality of cavities 306. In one embodiment, many analyte sensing members 308 may be printed onto a single substrate 322 which is then adhered to the cartridge to facilitate manufacturing and simplify assembly. The analyte sensing members 308 may be electrochemical in nature. The analyte sensing members 308 may further contain enzymes, dyes, or other detectors which react when exposed to the desired analyte. Additionally, the analyte sensing members 308 may comprise of clear optical windows that allow light to pass into the body fluid for analyte analysis. The number, location, and type of analyte sensing member 308 may be varied as desired, based in part on the design of the cartridge, number of analytes to be measured, the need for analyte sensing member calibration, and the sensitivity of the analyte sensing members. If the cartridge 300 uses an analyte sensing member arrangement where the analyte sensing members are on a substrate attached to the bottom of the cartridge, there may be through holes (as shown in FIG. 7), wicking elements, capillary tube or other devices on the cartridge 300 to allow body fluid to flow from the cartridge to the analyte sensing members 308 for analysis. In other configurations, the analyte sensing members 308 may be printed, formed, or otherwise located directly in the cavities housing the penetrating members 302 or areas on the cartridge surface that receive blood after lancing.

The use of the seal layer 320 and substrate or analyte sensing member layer 822 may facilitate the manufacture of these cartridges 10. For example, a single seal layer 320 may be adhered, attached, or otherwise coupled to the cartridge 300 as indicated by arrows 324 to seal many of the cavities 306 at one time. A sheet 322 of analyte sensing members may also be adhered, attached, or otherwise coupled to the cartridge 300 as indicated by arrows 325 to provide many analyte sensing members on the cartridge at one time. During manufacturing of one embodiment of the present invention, the cartridge 300 may be loaded with penetrating members 302, sealed with layer 320 and a temporary layer (not shown) on the bottom where substrate 322 would later go, to provide a sealed environment for the penetrating members. This assembly with the temporary bottom layer is then taken to be sterilized. After sterilization, the assembly is taken to a clean room (or it may already be in a clear room or equivalent environment) where the temporary bottom layer is removed and the substrate 322 with analyte sensing members is coupled to the cartridge as shown in FIG. 6. This process, allows for the sterile assembly of the cartridge, with the penetrating members 302 using processes and/or temperatures that may degrade the accuracy or functionality of the analyte sensing members on substrate 322. As a nonlimiting example, the entire cartridge 300 may then be placed in a further sealed container such as a pouch, bag, plastic molded container, etc . . . to facilitate contact, improve ruggedness, and/or allow for easier handling.

In some embodiments, more than one seal layer 320 may be used to seal the cavities 306. As examples of some embodiments, multiple layers may be placed over each cavity 306, half or some selected portion of the cavities may be sealed with one layer with the other half or selected portion of the cavities sealed with another sheet or layer, different shaped cavities may use different seal layer, or the like. The seal layer 320 may have different physical properties, such as those covering the penetrating members 302 near the end of the cartridge may have a different color such as red to indicate to the user (if visually inspectable) that the user is down to say 10, 5, or other number of penetrating members before the cartridge should be changed out.

FIG. 6 also shows that in some embodiments of the present invention, the layer 322 may optionally be removed and replaced by placing a plurality of analyte sensing members in a ring configuration 350 around the disc 300.

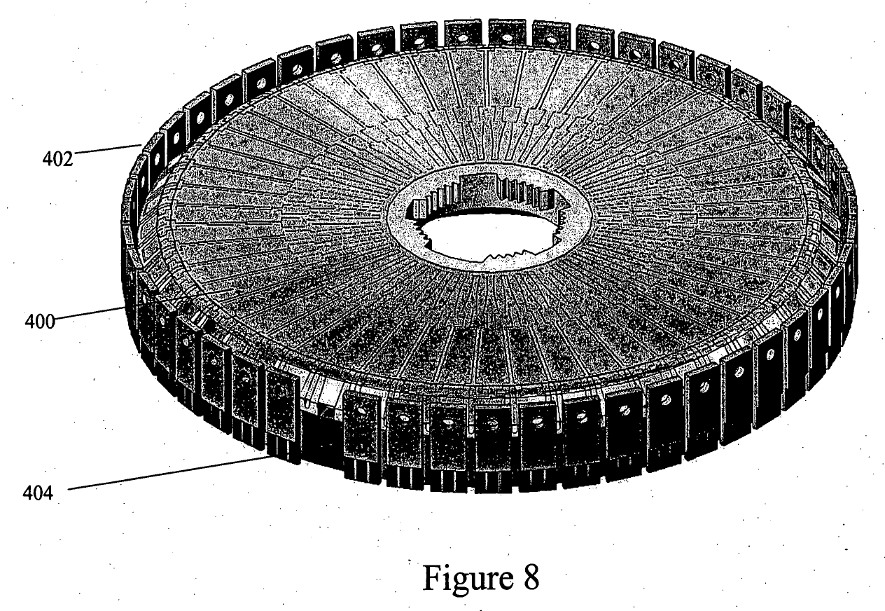

Referring now to FIGS. 7 and 8, another aspect of the present invention will now be described. FIG. 7 shows an penetrating member disc 400. In this concept, the inside of the penetrating member disc 400 is sealed from the external environment. Each cavity of the disc 400 is initially covered with a sterility barrier. The disc 400 may be surrounded by a plurality of test strip 402. The strips 402 may be three-dimensional devices which can capture and analyze a body fluid to measure analyte levels. In one embodiment of the present invention, the disc 400 may rest on top of a disc of desiccant 404. The desiccant will be used to absorb any excess humidity introduced by each body sample introduced into a test strip 402. FIG. 8 shows the disc 400 assembled with the test strips 402 and the desiccant disc 404.

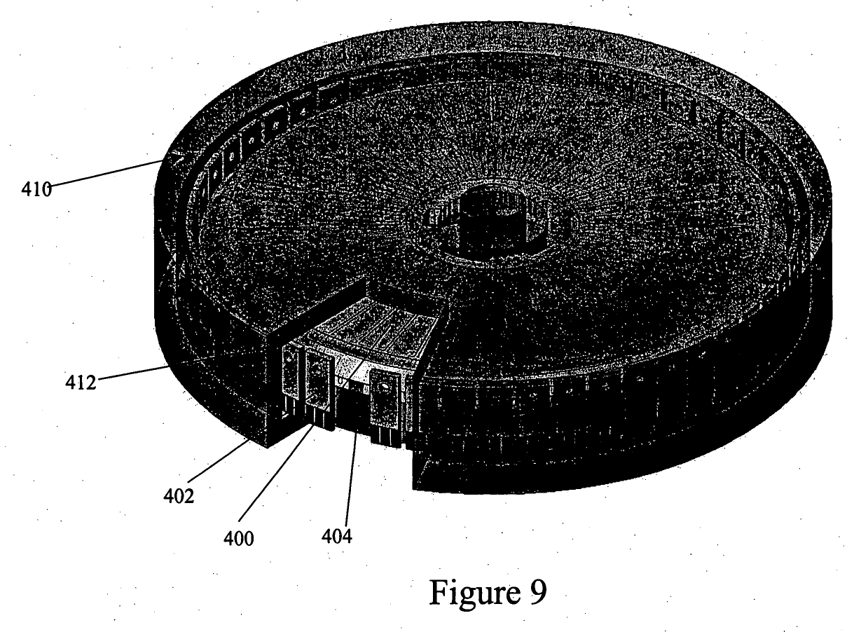

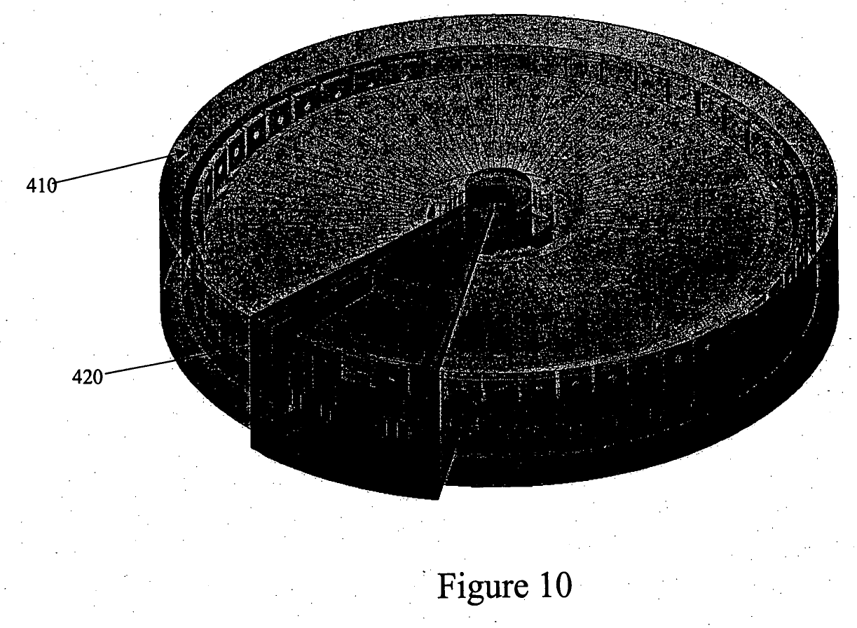

Referring now to FIG. 9, the device of FIG. 8 is now presented inside a cassette 410. The cassette will hold the penetrating member disc 400, the plurality of test strips 402, and the desiccant disc 404. The disc 400 may rotate so that a new, unused penetrating member maybe aligned with the opening 412 in the cassette 410. When the opening 412 is sealed as seen in FIG. 10, the environment inside the cassette 410 will be one that is sealed from the exterior atmosphere.

In one embodiment, the sealed cassette 410 will hold a disc 400 with 50 penetrating members and 50 test strips 402. The test strips 402 are not individually packaged. The interior of the cassette 410 is a sealed, desiccated environment. The cassette 410 is opened only during lancing and sample capture. The cassette 410 contains sufficient desiccant to keep the test strips 402 dry, even as blood or other body fluid is added during lancing and sample capture events.

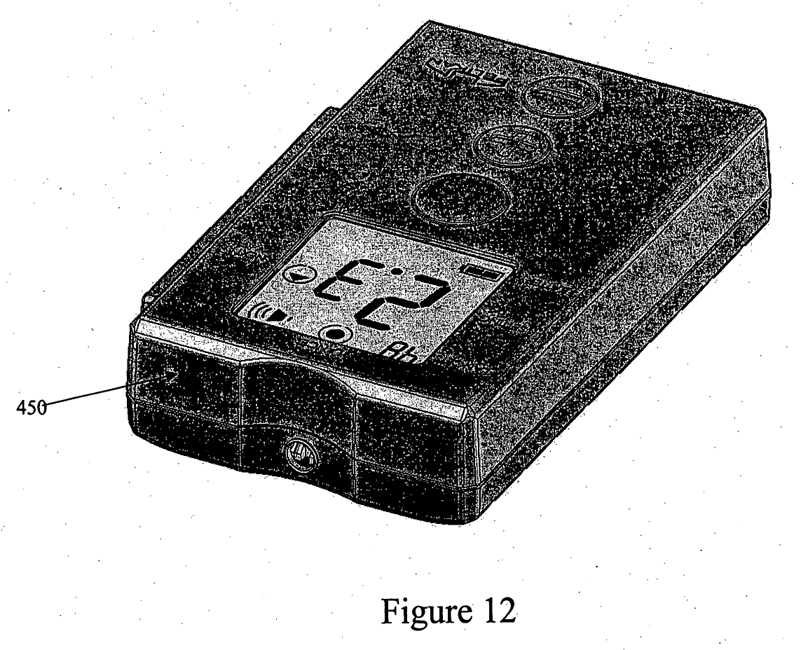

Referring now to the embodiment of FIG. 10, in this concept, only the cassette 410 is necessarily sealed. An access door or lid 420 is provided to open and close over the opening 412. The cassette 410 will be housed inside the instrument 450 shown in FIG. 12. A plurality of seals or gaskets are provided to seal the interface between access door 420 and the cassette 410 when the opening 412 is covered. The seal is broken only during lancing and blood sampling. An access door 420 covers the penetrating member exit port. In some embodiments, it should be understood that desiccant may incorporated into the cassette 410, and this desiccant dries the air inside of the cassette. Individual analyte sensing members in the disposable are not sealed from the interior environment of the cassette. However, since the test strips 402 are inside of the instrument 400, and the air inside the cassette 400 is kept dry, the analyte sensing members are still protected from humidity. The disposable may be similar to that shown in FIG. 6. Some embodiments may have a ring of analyte sensing members mounted on a scaffold around a disk that contains only penetrating members.

In various embodiments, the desiccant is present in an amount of no more than, 50 mm³, 10-20 mm^{3,} 10-15 mm^{3,} at least 1 mm³ per each of an analyte detecting member 16 and the like. The desiccant can be a variety of materials, including but not limited to, a molecular sieve, a silica gel, a clay, and the like. The molecular sieve can be mixed with a polymeric binder.

The plurality of analyte sensing members 308 can be supported on the scaffolding. The scaffolding can be attached to a bottom surface of the cartridge 300. The scaffolding can be made of a material such as, but not limited to, a polymer, a foil, and the like. The scaffolding can hold a plurality of analyte sensing members 308, such as but not limited to, about 10-50, 50-100, or other combinations of analyte sensing members 308. This facilitates the assembly and integration of analyte sensing members 308 with cartridge 300. These analyte sensing members 308 can enable an integrated body fluid sampling system where the penetrating members 14 create a wound tract in a target tissue, which expresses body fluid that flows into the cartridge 300 for analyte detection by at least one of the analyte sensing members 308.

In one embodiment, many analyte sensing members 308 can be printed onto a single scaffolding which is then adhered to the cartridge 300 to facilitate manufacturing and simplify assembly. The analyte sensing members 308 can be electrochemical in nature. The analyte sensing members 308 can further contain enzymes, dyes, or other detectors which react when exposed to the desired analyte. Additionally, the analyte sensing members 308 can comprise of clear optical windows that allow light to pass into the body fluid for analyte analysis. The number, location, and type of analyte detecting member 16 can be varied as desired, based in, part on the design of the cartridge 300, number of analytes to be measured, the need for analyte detecting member calibration, and the sensitivity of the analyte sensing members 308. Wicking elements, capillary tube or other devices on the cartridge 300 can be provided to allow body fluid to flow from the cartridge 300 to the analyte sensing members 308 for analysis. In other configurations, the analyte sensing members 308 can be printed, formed, or otherwise located directly in the cartridge 300.

In one embodiment, the desiccant material is external to the analyte sensing members 308. The desiccant can be on at least a portion of the analyte sensing members 308. In one embodiment, the scaffolding holds the desiccant. In another embodiment, the scaffolding includes a desiccant for each of an analyte detecting member 16. Each of analyte detecting member 16 can be stored in an air tight desiccated environment.

The desiccant can be molded and inserted into the scaffolding. In one embodiment, the desiccant and the scaffolding are co-molded simultaneously. In another embodiment, the scaffolding and the desiccant are co-molded sequentially. The desiccant can be present as a desiccant block inside of the instrument housing 10.

It should be understood that the cartridge 400 may rotate inside the cassette 410 and its motion is independent of that of the cassette 410. Additionally, in some embodiments, the cartridge 400 with the associated test strips 402 may be rotated so that a freshly used test strip 402 may be rotated to be next to a piece of desiccant that has not been previously associated with a used test strip. In this manner, the used test strip may be parked next to a piece of desiccant that has not been previously used to absorb humidity. This may involve rotating the cartridge 400 several or many positions away from the opening and is not merely a one increment rotation.

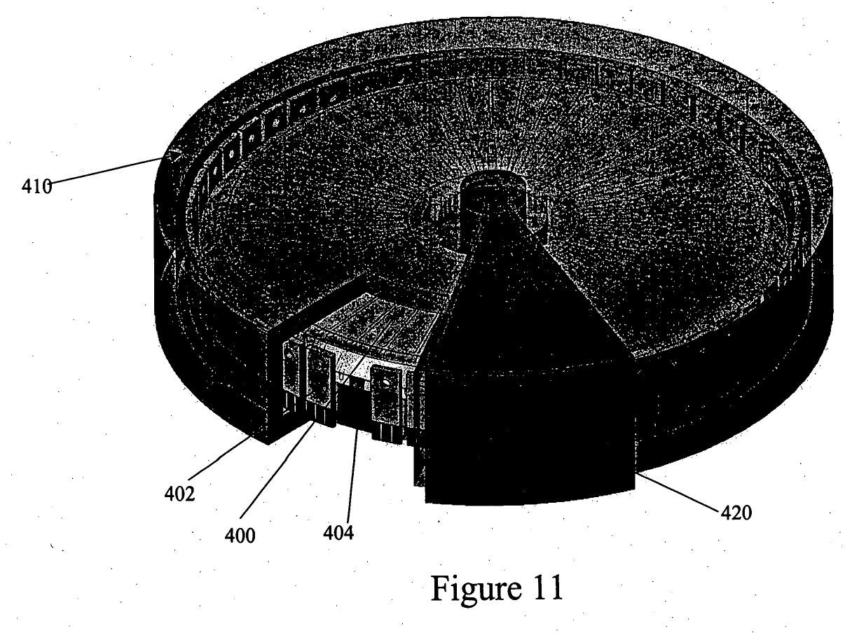

FIG. 11 shows the door or lid 420 swung to an open position where the user may access the test strip 402 and provide body fluid sample for analysis. In one embodiment, the size of the opening 412 may be such that only one test strip 402 is exposed at any one time when the door 420 is open. The opening 412 may also be sized to allow a gripper to access the penetrating member disc 400. The opening 412 may allow the gripper to engage the penetrating member and provide sufficient freedom of motion to move the penetrating member to pierce tissue.

FIG. 12 shows one embodiment of an instrument for use with the present invention.

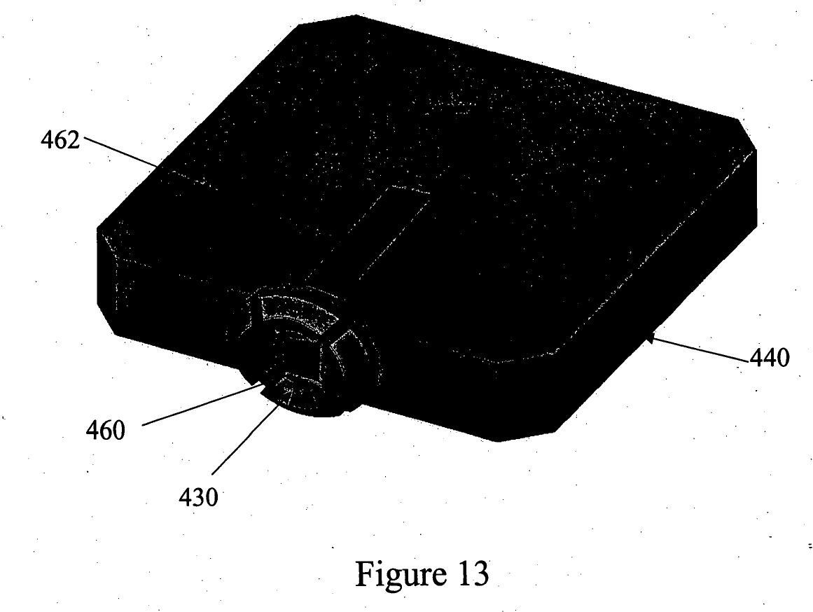

FIG. 13 shows yet another embodiment of the present invention wherein a front end 430 is incorporated on the outside of a more square cassette 440. The door 460 may also include a portion 462 that opens on the top to allow access to the cavities in the penetrating member disc 464. In one embodiment, the doors 460 and 462 are linked together so they open and close together.

A new cassette 410 is provided with each new disposable purchased by the user. The case is lined with or otherwise designed to contain desiccant in the cassette 410. In one embodiment, the desiccant may be designed to keep the analyte sensing members sufficiently dry for 90 days in a normal climate condition. Additionally, since every time the device is used is that a drop of blood is left inside the desiccated environment (on the analyte sensing member). An amount of desiccant sufficient to reduce the spike in humidity after each test is desired. In one embodiment, about 5 cc of desiccant is used. Other embodiments may use greater volumes to more quickly absorb the spike in humidity the occurs after blood is introduced into the desiccated environment. By way of example and not limitation, some embodiments may have 6 cc, 7 cc, 8 cc, 9 cc, 10 cc, 15 cc, 20 cc, 25 cc, 30 cc, or more of desiccant inside the cassette.

The lid or access door 420 may also include desiccant. If the device includes 1 mm thick desiccant layer, that is a significant amount of desiccant right there, in addition to the ˜15 cc in the disposable cassette.

The desiccant is replaced each time the disposable or cassette 410 is replaced by the user. A kit may be sold with instructions for use, a disposable with penetrating members (the disposable may also include a plurality of analyte sensing members), a desiccant (which by way of example and not limitation, depending on the embodiment may be part of the cassette 410, a separate block of desiccant to be placed inside the instrument, a desiccant lined case for placing the instrument inside, and/or a replacement lid with desiccant inside). It should be understood that the cassette 410 may be incorporated for use with any of the devices shown in U.S. Provisional Application Ser. No. ______ (Attorney Docket No. 38187-2766). This will incorporate a belt-and-suspender type concept where additional desiccant may be used in conjunction with that in the cassette 410.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, with any of the above embodiments, the shield or other punch may be adapted for use with other cartridges disclosed herein or in related applications. With any of the above embodiments, the methods for storage may be used with analyte sampling devices, analyte sampling and measurement devices, and/or analyte measurement devices. The use is not restricted. With any of the above embodiments, the lids may be flip up, rotated, or slide. They may be motorized or user actuated. With any of the above embodiments, the gasket between the door 420 and cassette 410 may also be designed for compression. The sliding lids are designed to compress the O-ring to provide a seal. It should be understood that for any of the embodiments above, instead of individual strips 402, they could be mounted on a tape and then positioned about the disc 400. In other embodiments, a tape of analyte sensing members is mounted about the disc 400.

The publications discussed or cited herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. All publications mentioned herein are incorporated herein by reference to disclose and describe the structures and/or methods in connection with which the publications are cited.

Expected variations or differences in the results are contemplated in, accordance with the objects and practices of the present invention. It is intended, therefore, that the invention be defined by the scope of the claims which follow and that such claims be interpreted as broadly as is reasonable.

1. A fluid sampling device comprising:
an instrument housing;
a cartridge defining a plurality of cavities, the cartridge sized to fit within the instrument housing; and
a cassette for housing the cartridge, the cassette sized to fit within the housing;
a plurality of penetrating members at least partially contained in the cavities of the cartridge wherein the penetrating members are slidably movable to extend outward from the cartridge to penetrate tissue, the cavities each having a longitudinal opening providing access to an elongate portion of the penetrating member;
a sterility barrier coupled to the cartridge, the sterility barrier covering a plurality of the longitudinal openings, wherein the sterility barrier covering the lateral openings is configured to be moved so that the elongate portion may be accessed by the gripper without touching the barrier; and
desiccant material inside the device to reduce humidity therein.

2. The device of claim 1, wherein the desiccant material is inside the cassette.

3. The device of claim 1, wherein each of a cavity is individually sealed with the sterility barrier.

4. The device of claim 3, wherein each cavity is individually sealed to provide that opening of one cavity does not interfere with the sterility in an adjacent or other cavity in the cartridge

5. The device of claim 1, wherein the sterility barrier is a planar structure adhered to a top surface of the cartridge.

6. The device of claim 1, further comprising:
a plurality of analyte detecting members, each of an analyte detecting member coupled to a penetrating member.

7. The device of claim 6, wherein the desiccant is present in an amount of no more than 50 mm3 per each of an analyte detecting member.

8. The device of claim 6, wherein the desiccant is present in an amount of 10-20 mm3 per each of an analyte detecting member.

9. The device of claim 6, wherein the desiccant is present in an amount of 10-15 mm3 per each of an analyte detecting member.

10. The device of claim 6, wherein the desiccant is present in an amount of at least 1 mm3 per each of an analyte detecting member.

11. The device of claim 1, wherein the desiccant is selected from at least one of a molecular sieve, a silica gel or a clay.

12. The device of claim 11, wherein the molecular sieve is mixed with a polymeric binder.

13. The device of claim 7, further comprising
a scaffolding that supports the plurality of analyte detecting members.

14. The device of claim 13, wherein the scaffolding holds the desiccant.

15. The device of claim 14, wherein the scaffolding includes a desiccant for each of an analyte detecting member.

16. The device of claim 1, wherein the desiccant is present as a desiccant block inside of the instrument housing.

17. The device of claim 14, wherein the desiccant is molded and inserted into the scaffolding.

18. The device of claim 13, wherein the desiccant is coupled with the scaffolding.

19. The device of claim 13, wherein the desiccant and the scaffolding are co-molded simultaneously.

20. The device of claim 13, wherein the scaffolding and the desiccant are co-molded sequentially.

21. The device of claim 20. wherein the desiccant material is configured to be replaced when the cartridge is replaced from the instrument housing.

22. The device of claim 16, wherein the desiccant material is external to the analyte detecting members.

23. The device of claim 16, wherein the desiccant is on at least a portion of the analyte detecting members.

24. The device of claim 7, wherein each of an analyte detecting members are stored in an air tight desiccated environment.

25. The device of claim 7, wherein an air seal is formed around each of an analyte detecting member.

26. The device of claim 1, wherein an air tight seal is formed around the cartridge.

27. The device of claim 1, wherein an air tight seal is formed around the instrument housing.

28. The device of claim 1, wherein the instrument housing is in a sealed case.

29. The device of claim 1, further comprising:
a device that provides controlled velocity and depth of penetration of the penetrating members.

30. A device for use in penetrating tissue to obtain a body fluid sample, comprising:
an instrument housing;
a cartridge; and
a plurality of penetrating members slidably coupled to the cartridge, each of the penetrating members having a distal end sufficiently sharp to pierce tissue and each of the penetrating members being moveable relative to the other ones of the penetrating members, so that the distal end of the respective penetrating member is movable to penetrate tissue;
wherein each of the penetrating member is a bare lancet does not penetrate an outer sterility barrier during actuation;
a plurality of analyte sensing members mounted about the instrument housing;
a cassette to contain the cartridge and sized to fit within the instrument housing, the cassette providing a sealed environment when a lid on the cassette is closed to improve the storage condition of the analyte sensing members; and
desiccant in the device.

31. The device of claim 30, wherein the cassette contains desiccant.

32. The device of claim 30, wherein each of a cavity is individually sealed with the sterility barrier.

33. The device of claim 32, wherein each cavity is individually sealed to provide that opening of one cavity does not interfere with the sterility in an adjacent or other cavity in the cartridge

34. The device of claim 30, wherein the sterility barrier is a planar structure adhered to a top surface of the cartridge.

35. The device of claim 30, wherein each of an analyte detecting member is coupled to a penetrating member.

36. The device of claim 30, wherein the desiccant is present in an amount of no more than 50 mm3 per each of an analyte detecting member.

37. The device of claim 30, wherein the desiccant is present in an amount of 10-20 mm3 per each of an analyte detecting member.

38. The device of claim 30, wherein the desiccant is present in an amount of 10-15 mm3 per each of an analyte detecting member.

39. The device of claim 30, wherein the desiccant is present in an amount of at least 1 mm3 per each of an analyte detecting member.

40. The device of claim 30, wherein the desiccant is selected from at least one of a molecular sieve, a silica gel or a clay.

41. The device of claim 40, wherein the molecular sieve is mixed with a polymeric binder.

42. The device of claim 30, further comprising
a scaffolding that supports the plurality of analyte detecting members.

43. The device of claim 42, wherein the scaffolding holds the desiccant.

44. The device of claim 43, wherein the scaffolding includes a desiccant for each of an analyte detecting member.

45. The device of claim 30, wherein the desiccant is present as a desiccant block inside of the instrument instrument housing.

46. The device of claim 43, wherein the desiccant is molded and inserted into the scaffolding.

47. The device of claim 42, wherein the desiccant is coupled with the scaffolding.

48. The device of claim 42, wherein the desiccant and the scaffolding are co-molded simultaneously.

49. The device of claim 42, wherein the scaffolding and the desiccant are co-molded sequentially.

50. The device of claim 49, wherein the desiccant material is configured to be replaced when the cartridge is replaced from the instrument instrument housing.

51. The device of claim 47, wherein the desiccant material is external to the analyte detecting members.

52. The device of claim 47, wherein the desiccant is on at least a portion of the analyte detecting members.

53. The device of claim 30, wherein each of an analyte detecting members are stored in an air tight desiccated environment.

54. The device of claim 30, wherein an air seal is formed around each of an analyte detecting member.

55. The device of claim 30, wherein an air tight seal is formed around the cartridge.

56. The device of claim 30, wherein an air tight seal is formed around the instrument housing.

57. The device of claim 30, wherein the instrument instrument housing is in a sealed case.

58. The device of claim 30, further comprising:
a device that provides controlled velocity and depth of penetration of the penetrating members.

59. A method comprising:
providing an analyte sampling device having a instrument housing and a cartridge having a plurality of penetrating members wherein the penetrating members are slidably movable to extend outward from lateral openings on the cartridge to penetrate tissue;
providing the cartridge in a sealed cassette containing desiccant, wherein the cassette has a lid that is opened when the cartridge is about to be used.
